(19) United States
(12) Patent Application Publication
(10) Pub. No.: US 2014/0186828 A1
(43) Pub. Date: Jul. 3, 2014

(54) METHODS FOR DETERMINING CELL VIABILITY USING MOLECULAR NUCLEIC ACID-BASED TECHNIQUES

(71) Applicant: Shawn Mark O'Hara, Richboro, PA (US)

(72) Inventor: Shawn Mark O'Hara, Richboro, PA (US)

(21) Appl. No.: 13/977,719

(22) PCT Filed: Dec. 27, 2011
(86) PCT No.: PCT/US11/67329

§ 371 Date: Feb. 27, 2014

Related U.S. Application Data
(60) Provisional application No. 61/428,892, filed on Dec. 31, 2010.

(51) Int. Cl. C12Q 1/68

(52) U.S. Cl. CPC C12Q 1/689
USPC 435/6.11; 435/6.12

(57) ABSTRACT

The present invention relates to novel methods, and kits, for selectively excluding dead cells from a mixture containing live and dead cells, such as microbe cells in clinical samples, blood products, medical/biotechnology products and food products where subsequent interrogation of the selected live cells are an indicator of the presence of microbe viability. In particular, the invention relates to improved methods for performing direct nucleic acid amplification techniques such as Polymerase Chain Reaction (PCR) and isothermal techniques in blood and other body fluids, for correlation with microbe cell viability from Bacteremia and Fungemia samples. The improved methods provided by the invention are particularly advantageous for the diagnosis of septicemia and to determine pathological conditions in all other normally sterile body fluids.

### CROSS REFERENCE TO RELATED APPLICATION

This application is a non-provisional application, which is incorporated by reference herein and claims priority of U.S. Provisional Application No. 61/428,892, filed Dec. 31, 2010.

### BACKGROUND OF THE INVENTION

1. Technical Field

The present invention relates to methods for selectively excluding, from molecular detection, DNA of dead cells from a mixture containing live and dead cells, and in particular relates to improved methods for performing direct Polymerase Chain Reaction (PCR) techniques in blood and other body fluids for correlation with viable microbe cells from Bacteremia, Fungemia, Viremia and other types of parasite containing samples. The improved methods provided by the invention are particularly advantageous for the diagnosis of septicemia.

2. Background Art

In diagnosing septicemia the time to result (TTR) is the most important determination of patient survival. Currently, blood culture is the gold standard, but is relatively slow, generating viable microorganisms for subsequent identification with a approximate median time of 15 hours (in the general range of 3 hours to 5 days) to turn positive, after which microbe identification typically can add another 1-2 days for the analysis. Molecular methods such as PCR offer vastly improved TTR for microbe identification, but suffer from a lack of specificity primarily due to inadequate selectivity of viable microbe cells during sample preparation. Traditional septicemia PCR testing of blood conventionally requires costly DNA isolations to remove PCR inhibitors, but isolation also causes false positives and loss of sensitivity compared to the gold standard of blood culture, primarily due to the inclusion of DNA from dead microbe cells and sample processing dependent losses during the DNA isolation procedure.

Traditionally, septicemia blood sample PCR preparations have always isolated DNA from blood and blood products to remove the long and well known blood derived PCR Inhibitors of Taq polymerases (see the Klouche and Schroder article cited below). Recently in an attempt to overcome this inhibition some groups have developed PCR-enhancing mixtures as well as modified thermal-stable polymerases (for example, the well-known “omni taq” and “Phusion” techniques) engineered to reduce the inhibitory affect of blood products on these polymerases (see JMD, 2010; 12(2), pp. 152-161). However the constraints of both of these approaches still suffer from either a lack of sensitivity due to low tolerated blood volume, and the high costs and loss of sample and high complexity that are associated with isolation systems. Furthermore DNA Isolation systems often include the cell free DNA from dead cells, which can have the effect of causing confounding false positives.

Klouche, M. and Schroder, U. in an article entitled “Rapid methods for diagnosis of bloodstream infections,” published in Clin. Chem. Lab. Med., 2008; 46(7), pp. 888-908, disclose that direct nucleic acid-based detection and identification of microbial pathogens in blood from patients can be a promising tool for rapid diagnosis of bloodstream infections. According to this article, the significance of detection of circulating bacterial or fungal nucleic acids by broad-range molecular approaches for routine workup of bloodstream infections, however, is at present not clear. Encouraging issues for improvement of quality and reproducibility of molecular diagnostic applications in bloodstream infections include selective enrichment procedures for bacterial nucleic acids, blocking or elimination methods of excess human DNA, and use of viability markers to discriminate clinically relevant findings, as shown in experience from microbial safety analysis. Despite the currently expensive and technically demanding technologies, disease-oriented multiplex PCR, pathogen microarrays and proteomic profiling have the potential to evolve as important rapid and high-throughput diagnostic means for infectious disease diagnosis. At present, three main considerations preclude the unique application of molecular technologies in routine diagnosis of bloodstream infections: the difficulties in interpretation of the NAT results due to 1) the high risk of external contamination, the extended persistence of nucleic acids after infection, and transient bacteraemia, 2) the limited analytical sensitivity for clinically relevant low bacterial loads, and for detection of certain bacteria and fungi, and 3) the lack of routine antimicrobial susceptibility testing by molecular as well as by proteomic testing.

Differentiation of live and dead cells is an important challenge in microbial diagnostics. Metabolic and reproductive activity, and, in the case of pathogenic microorganisms, the potential health risk are limited to the live portion of a mixed microbial population. Four physiological states are used in the conventional art to distinguish, in flow cytometry using fluorescent stains: reproductively viable, metabolically active, intact and permeabilized cells. Depending on the conditions, all stages except the permeabilized cells can have the potential of recovery upon resuscitation and thus have to be considered potentially live. Due to the relatively long persistence of DNA after cell death in the range between days to 3 weeks, DNA-based diagnostics tend to overestimate the number of live cells. DNA extracted from a sample can originate from cells in any of the four mentioned physiological states including the dead permeabilized cells. Detection of the latter, however, is not desired. The most important criterion for distinguishing between viable and irreversibly damaged cells is membrane integrity. Sorting out noise derived from membrane-compromised cells helps to assign metabolic activities and health risks to the intact and viable portion of bacterial communities. Live cells with intact membranes have been distinguished by their ability to exclude DNA-binding dyes that easily penetrate dead or membrane-compromised cells.

Recently, EMA-PCR was reported to be an easy-to-use alternative to microscopic or flow-cytometric analyses to distinguish between live and dead cells. This diagnostic DNA-based method combines the use of a live-dead discriminating dye with the speed and sensitivity of real-time PCR. Ethidium monoazide (EMA). a DNA-intercalating dye with the azide group allowing covalent binding of the chemical to DNA upon exposure to bright visible light (maximum absorbance at 460 nm), has been used in this regard. Cells are exposed to EMA for 5 minutes allowing the dye to penetrate dead cells with compromised cell walls/membranes and to bind to their DNA. Photolysis of EMA using bright visible light produces a nitrene that can form a covalent link to DNA and other molecules.

Photo-induced cross-linking has been reported to inhibit PCR amplification of DNA from dead cells. It has been recently shown that EMA-crosslinking to DNA actually render the DNA insoluble, and leads to loss together with cell debris during genomic DNA extraction. Unbound EMA, which remains free in solution, can be simultaneously inactivated by reacting with water molecules. The resulting hydroxylamine is no longer capable of covalently binding to DNA. DNA from viable cells, protected from reactive EMA before light-exposure by an intact cell membrane/cell wall, is therefore not affected by the inactivated EMA after cell lysis. Therefore, EMA treatment of bacterial cultures comprised of a mixture of viable and dead cells thus leads to selective removal of DNA from dead cells. The species tested were E. coli 0157:H7, Salmonella typhimu\acute{η}iim, Listeria monocytogenes and Campylobacter Jejuni. These studies did not examine, however, the selective loss of DNA from dead cells.

Though this technique is promising, the use of EMA prior to DNA extraction has been found to suffer from a major drawback. In some cases, the treatment also resulted in loss of approximately 60% of the genomic DNA of viable cells harvested in log phase. It has been observed that EMA also readily penetrates viable cells of other bacterial species resulting in partial DNA loss. This lack of selectivity and of overall applicability has led to testing of a newly developed alternative chemical: Propidium monoazide (PMA). In a published patent application, WO/2007/100762 to Nocker, et al., published Sep. 7, 2007, there is disclosed the suitability of PMA to selectively remove detection of genomic DNA of dead cells from bacterial cultures with defined portions of live and dead cells. PMA is identical to propidium iodide (PI), except that the additional presence of an azide group allows crosslinkage to DNA upon light-exposure. PI has been extensively used to identify dead cells in mixed populations. The higher charge of the PMA molecule (2 positive charges compared to only one in the case of EMA) and because selective staining of nonviable cells with PI had been successfully performed on a wide variety of cell types, led those in the field to believe that the use of PMA might mitigate the drawbacks observed with EMA. In this published patent, PMA concentration and incubation time were optimized with one gram-negative and one gram-positive organism before applying these parameters to the study of a broad-spectrum of different bacterial species. The disclosed method purportedly limits molecular diagnostics to the portion of a microbial community with intact cell membranes. This is achieved by exposing a mixture of intact and membrane-compromised cells to a phenanthridium derivative. In a disclosed preferred embodiment, PCR is performed using genomic DNA from the mixture as a template.

Also, Published U.S. Patent Application No. 2008/0160528, to Lorenz, published Jul. 3, 2008, discloses the use of nucleases, especially DNA-degrading nucleases, for degrading nucleic acids in the presence of one or several chaotropic agents and/or one or several surfactants. This patent application further discloses a method for purifying RNA from mixtures of DNA and RNA as well as kits for carrying out such a method. Also disclosed is a method for specifically isolating nucleic acids from microbial cells provided in a mixed sample which additionally comprises higher eukaryotic cells as well as kits for carrying out such a method.

Another published patent application, WO/2001/077379 to Rudi, et al., published Oct. 18, 2001, discloses methods of detecting cells in a sample and for obtaining quantitative information about cell populations within a sample. In particular, a method is disclosed for distinguishing between living and dead cells in a sample. The method comprises contacting the sample with a viability probe which modifies the nucleic acid of dead cells within the sample, and detecting nucleic acid from the cells in the sample. Also described is a method of detecting cells in a sample, the method comprising: (a) contacting the sample with a viability probe which labels the nucleic acid of dead cells within the sample; (b) separating the nucleic acid from the cells into labeled and non-labelled fractions; and (c) detecting the nucleic acid in one or both of the fractions.

### SUMMARY OF THE INVENTION

In view of the foregoing background art, it can be seen that a paradigm shift would be to develop a method that effectively discriminates live vs. dead microbe cell DNA prior to molecular nucleic-acid based analysis techniques (for example before PCR set up), and that also circumvents the costly negative effects of traditional isolation designed to remove, e.g., PCR inhibitors and concentrate target DNA. Surprisingly, in accordance with the practice of an embodiment of the present invention, it has been shown that PCR correlates with viable microbe cells derived from blood, employing a combination of selective blood cell lysis, washing (and or) DNase along with subsequent microbe cell lysis and PCR.

Thus, in contrast to the conventional methods described above, the present invention seeks to realize the potential TTR advantage of molecular nucleic-acid based techniques, including PCR, by dramatically simplifying costly DNA isolations and sample preparation, and by not isolating DNA, but rather by performing a rapid and simple direct-analysis on crude microbe lysates after a rapid separation of the dead microbe DNA and cells, resulting in the selective enrichment of viable microbe cells. This is particularly and unexpectedly advantageous in the diagnosis of septicemia, and is accomplished according to a preferred embodiment of the present invention by:
I. The removal of confounding dead microbe cell DNA prior to a positive non contaminated PCR result indicates that viable cells are present, and as such the PCR result will indicate the presence of viable septicemia microbe(s), i.e., blood microbe PCR=viable septicemia microbes.
II. As is well known, dead microbe cells from blood cannot grow in blood culture, thus any two or more time points measuring significant microbe-specific PCR signal increases from a single blood culture bottle must be measuring viable microbes.
III. PCR inhibitors from blood can be eliminated via a simple combination of chemical denaturants (chaotropes: detergents, pH, salts, organic chemical based differential salvation via dipole moment such as alcohols and amine containing compounds & enzymes such as nucleases, proteinases etc.) and washing, thereby circumventing DNA isolation and enabling microbe lysate-Direct-PCR.
IV. The ratio of live/dead microbes present in blood and blood culture can then be used as a measure of the effectiveness of a therapy and of testing the efficacy of treatment.

Accordingly, it is an objective of the present invention to provide improved methods for selectively excluding, from molecular detection, DNA of dead cells from a mixture containing live and dead cells.

It is a further objective of the of the present invention to provide improved methods that effectively discriminate live vs. dead microbe cell DNA prior to molecular nucleic-acid based analysis or PCR set up, and that also circumvents the costly negative effects of traditional isolation such as those designed to remove PCR inhibitors and concentrate target DNA.

It is another objective of the present invention to provide methods of correlating results of PCR and other molecular analysis techniques with the presence of viable microbe cells derived from blood, for example by employing a combination of selective blood cell lysis, washing (and or) DNase along with subsequent microbe cell lysis and PCR.

It is yet another objective of the present invention to provide improved methods for performing direct PCR techniques in blood and other body fluids for correlation with viable microbe cells from Bacteremia and Fungemia samples, such improved methods provided by the invention being particularly advantageous for the diagnosis of septicemia.

Further objectives and advantages of the present invention will be apparent from the following description of preferred embodiments thereof.

### BRIEF DESCRIPTION OF THE DRAWINGS

FIG. 1 shows, in table form, the results of experiments conducted to compare filter-bead mill-in situ microbe lysis and analyte analysis via DNA Polymerase (PolMA), and genomic DNA via quantitative gene specific PCR.

FIG. 2 shows an illustration in diagram form of a strategy for detection of microbes in lysates according to the invention.

FIG. 3 shows flow diagrams illustrating that the addition of trypsin and DNase enables significant reduction of clogging observed during the processing of two “difficult” clinical samples in accordance with the present invention.

### DETAILED DESCRIPTION OF THE INVENTION

Although the present invention has been described, the following examples are also provided by way of specific illustration of embodiments of the invention and for purposes of clarity of understanding. It will be readily apparent to those of ordinary skill in the art, in light of the teachings of this invention as set forth herein, that certain changes and modifications may be made to these embodiments thus described without departing from the spirit or scope of the invention.

A chaotropic agent, also known as chaotropic reagent and chaotrope, is a substance which disrupts the three dimensional structure in macromolecules such as proteins, DNA, or RNA, and denatures them. Chaotropic agents interfere with stabilizing inter-molecular interactions mediated by non-covalent forces such as hydrogen bonds, van der Waals forces, and hydrophobic effects. Often structural features, as detected by means such as circular dichroism can be titrated in a chaotrope concentration-dependent fashion. Chaotropic reagents include, for example:

Urea 6-8 mol/l

Guanidinium chloride 6 mol/l

Lithium perchlorate 4.5 mol/l

Denaturation (biochemistry)

In addition, high generic salts can have chaotropic properties, by shielding charges and preventing the stabilization of salt bridges. Hydrogen bonding is stronger in nonpolar media, so salts, which increase the dipole moment of the solvent, can also destabilize hydrogen bonding.

Often structural features, as detected by means such as circular dichroism can be titrated in a chaotrope concentration-dependent fashion. Some examples of historically useful chaotropic reagents in biochemistry and molecular biology include: Urea 6-8 mol/l, guanidinium chloride 6 mol/l, lithium perchlorate 4.5 mol/l, alcohols, amines (especially quaternary amines), detergents (especially nonionic), pH change, betaine, proline, carnitine, trehalose, NP-40 and the like, as well as BSA. In accordance with the present invention the design of experiment (DoE) process has been used for optimization of effective formulation ranges and combinations of ranges of various chaotropes (mixtures or reagents, or “cocktails”) to: a) denature dead cell structures such that they are easily separated from live cells based on their size (filtration) and density (centrifugation); and b) create resultant chaotrope cocktail exposed live cell separated solutions that are directly compatible with downstream analysis amplification assays, such as PCR and the live cell derived endogenous proteins, and that maintain their measurable biochemical activities. Effectively the chaotropic cocktails will be optimized to differentiate live from dead cells based on the differential membrane integrity thereof, maintaining live cell endogenous protein activities for viability correlation analysis.

Sample Preparation:

Preferential blood cell lysis conditions yield preferential homogenization of blood cells from blood-microbe mixtures such as found in septicemia blood culture samples. Homogenization needs to occur at a sufficient level (creating a fluid) which enables passage of unwanted blood cells fluid through a filter from the Feed side (retaining desired microbe cells) through to the filtrate side effectively separating these two populations. These lysis conditions would enable the microbial cells to remain intact and thus enable rapid/sensitive filter-based separation of homogenized blood cells by retaining microbe cells.

In accordance with the present invention, differential blood cell Lysis and sufficient homogenization of their resulting cell debris are employed to reduced blood cells down to a fluid level enabling differential filterability where the filter retains microbes on the Feed side, thus separating the intact microbes, for subsequent sterile fluids analyses. Filter pore sizes known to those in the art as pore sizes measuring between 0.45 um, 0.22 um, 0.1 um in diameter should be sufficient. However these effective pore sizes could be both smaller than 0.1 and larger than 0.45 depending on the microbe and differential cell debris size filterability. Conditions include but are not limited to optimized combinations of detergent, proteinases, chaotrops, denaturants, and nucleases to achieve the desired effects.

Microbe specific filter-in situ is defined herein as employing physical and biochemical cell wall lysis methods while microbes are captured on the Feed side of the filter and/or subsequent microbe specific analyte assays applied in situ. Furthermore, herein “in situ” means lysis and or subsequent analysis occurs after differential separation of undesired interfering cells (i.e. Blood cells) while desired microbe cells are still retained on the Feed side of the filter. Thus it is expected that the captured microbes are likely suspended in residual Feed Filter solution used to load and wash the filter. The physical forces employed to lyse these now separated, intact and filter-contained microbes are those common to those skilled in this art including but not limited to enzymatic cell wall digestion. Furthermore in accordance with the invention filter-in situ sonication of all microbes by direct probe contacting the residual liquid retained by surface tension on the filter side containing the separated microbes, alternatively by sonic probe contacting the opposite side of the filter from the microbes and transferring its lytic energy via through the pores not through the solid filter material. In addition, it has been surprisingly found that efficiency of filter-bead-mill in situ for microbe lysis of bacteria and yeast occurs as well in a closed microfuge tube as it does directly on the filter Feed surface after capturing microbes spiked in blood where the blood cells were differentially lysed and filter separated. In this manner filter-in situ as defined herein is an elegant simplification of septicemia sample preparation enabling more efficient processing with less manipulations, less potential for contamination, more flexible formats both manually and for automated device designs.

As used in the following examples, filtration is employed as the term is commonly used in the art, that is, a mechanical or physical operation which is used for the separation of solids from fluids (liquids or gases) by interposing a medium through which only the fluid can pass. In a typical simple filtration, oversize particles in the liquid being filtered cannot pass through the lattice structure of the filter, whereas fluid and small particles pass through, becoming filtrate.

### Example 1

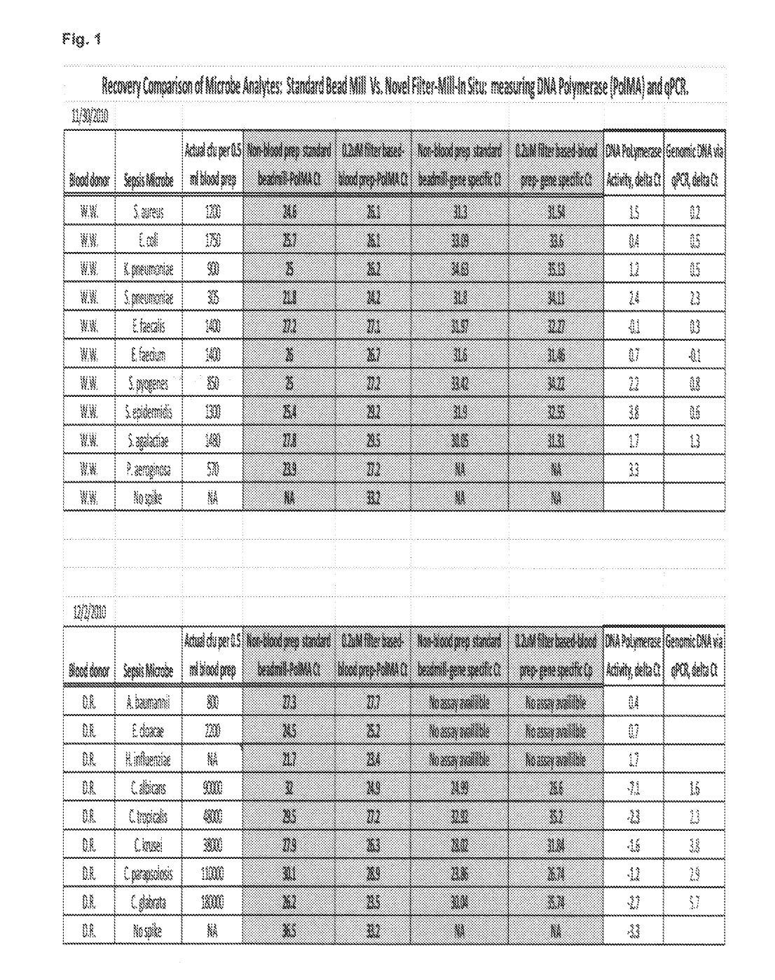

Experiments were conducted to compare filter-bead mill-in situ microbe lysis and analyte analysis via DNA Polymerase (PolMA), and genomic DNA via quantitative gene specific PCR. The results are presented in the tables illustrated in FIG. 1 of the drawings.

Interpretation of delta Ct values must be greater than two to be considered a significant difference when comparing relative qPCR values as is done here.

### Results and Conclusions:

The relative qPCR difference values between starting input microbe spikes and corresponding filter captured samples shows in general a very high % recovery of various microbes spiked into blood and then captured on the Feed side of the filter and then bead mill lysed on the feed side of the filter termed here “filter-mill in situ”. Of the 14 different microbes that were measurable by PCR only four (all Candida yeasts) (28%) showed any significant PCR recovery differences. Yet for these yeasts there was an increase in measurable DNA polymerase activity from these same samples. Overall, this indicated an excellent recovery and high efficiency filter mill in situ yielding both high DNA polymerase activity and amplifiable genomic DNA. Unexpectedly, significant negative values in bold red show that filter in situ dependent PolMA in accordance with the invention can be a significant improvement standard milling in a microfuge tube.

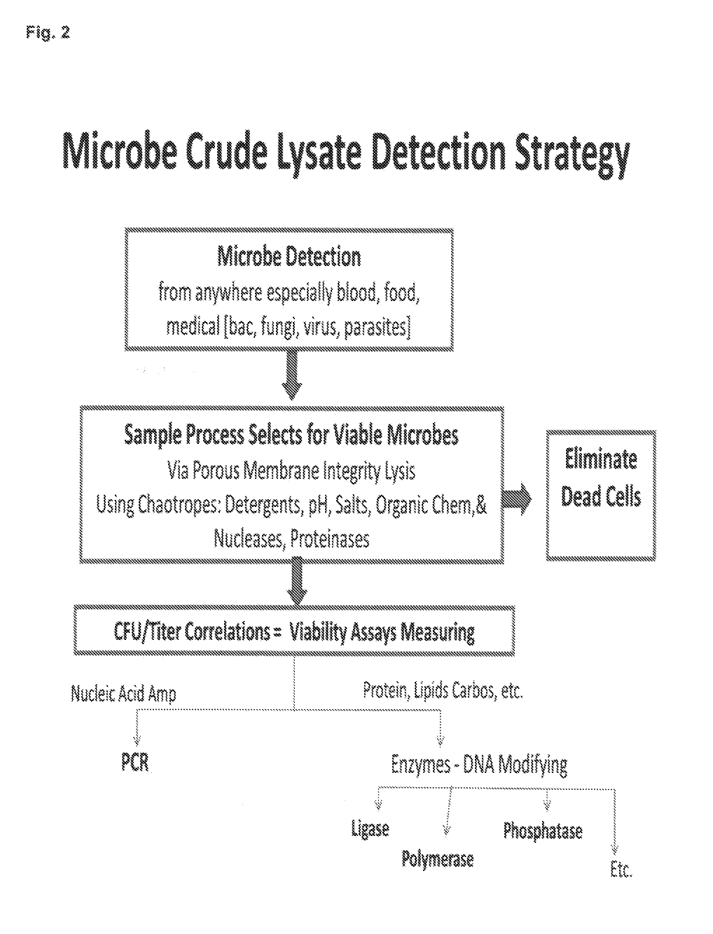

The strategy for detection of microbes in lysates according to the invention can be summarized in the diagram appended hereto as FIG. 2.

### Example 2

This example of an embodiment of the invention demonstrates the suitability of the present invention for circumventing the necessity for conventional DNA isolation techniques, and for enabling microbe lysate-direct-probe-based-PCR techniques to be performed
a. Staphylococcus Aureus (SA) was spiked into standard blood cultures, (Candida consensus assay, E. Coli, E faecium) followed by WBC detergent+base lysis, pelletizing, and washing.
b. It was found that after direct lysate PCR using both TaqMan probe and SYBR that the direct probe procedure in accordance with the invention was in each case superior in terms of higher tolerance of % lysate in PCR (up to 17% with no inhibition detected from at least 5000 microbes in 5 ul mill lysate, in 30 ul PCR. Blood culture positive bottles will contain 4000 microbes/ml of culture, placing 2 ml in prep yields 8000 microbes/50 ul lysate of which 5 ul in 30 ul PCR reaction=160 microbes in PCR (Upper BC level required assay tolerance). It is presently estimated that the limit of detection of BC to be 500 microbes/bottle or 10 microbes/ml, therefore 5 ul=2. If 10 microbes/bottle (common), then 5 ul=0.2 microbes then requiring 6 doubling generations to =640/bottle, which can be detectable.
c. Accordingly, it has been shown in accordance with the invention that SA microbes run through (chaotrope+detergent) MolYsis buffer and DNase treatment, followed by 1 TE pellet & wash are compatible with mill-direct probe PCR. The novel improved methods of the invention were shown by the improvements in the blood mill direct system utilized, in terms of sensitivity and tolerance of % blood over the conventional art, by comparing blood culture bead mill systems without denaturants (the Becton Dickinson Staph S/R kit, commercially available from Becton Dickinson), where only 1/10e6th of sample is in PCR, to the system provided by the improvements of the present invention with denaturants (DoE: guanidine/tween, trition/NaOH, tween/trition etc.)

### Example 3

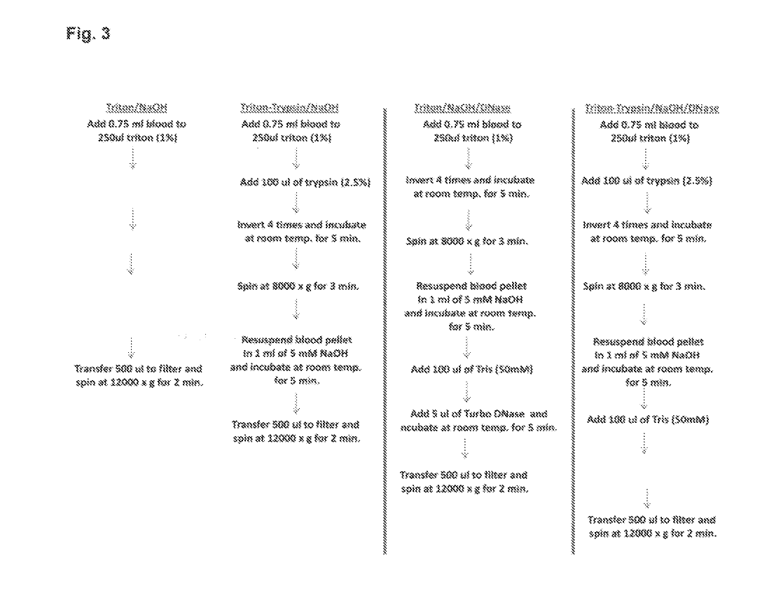

Further in experiments during the development of the invention, it was demonstrated that the addition of trypsin and DNase enables significant reduction of clogging observed during the processing of two “difficult” clinical samples in accordance with the present invention, as presented in the flow diagrams shown in FIG. 3 appended hereto.

It will be appreciated by those of ordinary skill in the art that the broad fundamental principles and teachings of the present invention are capable of being applied to optimize all variations of denaturant-enabled-crude lysate (bead mills & ultrasonics)-direct-probe/SYBR-PCR analysis of various biological tissue samples (including, but not limited to, blood, body fluid, and soft tissues) for not only SA as specifically described above, but also for various pathogens, such as any bacteria, fungi, virus, parasites, etc.

The above examples also show that the practice of the methods provided by the invention can efficiently suppress signals from killed cells in defined mixtures or in an environmental sample spiked with defined mixtures of live and killed cells. It is also worthwhile to note that treatment of samples in accordance with the invention might be a good way to exclude membrane-compromised cells from analysis.

Summarizing the above, this invention provides novel methods enabling fast and easy-to-perform pre-treatment of a bacterial population before further downstream analyses. Although the potential numerous applications of the invention will be appreciated by those skilled in the art, the methods provided by the invention may have a great impact on DNA-based diagnostics in various fields, including pathogen diagnostics, bioterrorism and microbial ecology.

In the practice of a preferred embodiment of the invention, it will be apparent that because cells don't grow, any PCR measurement of at least two separate time points using separate but equal aliquots from a single blood culture that shows a significant increase in a microbe target signal must be due to microbe growth, thereby indicating the presence of viable microbes (disregarding contamination effects). It is to be appreciated that non-growth based single point positive PCR analysis of blood will indicate the presence of a viable microbe when all dead cell DNA has been eliminated, prior to viable microbe lysis and PCR setup baring any PCR process induced contamination. This can be demonstrated by by DNasing and Washing away dead cell DNA.

Although specific references are made herein to PCR, It is further to be appreciated that the improvements of the present invention are not limited to PCR or similar methodologies. Amplification assays contemplated for use in the present invention include, but are not limited to, other well-known nucleic-acid based techniques such as DNA amplification assays, PCR assays incorporating thermostable polymerases, and isothermal amplifications methods. It is to be appreciated that one skilled in the art may conceive of various suitable amplification methods that will be useful in the practice of the present invention, and that therefore the invention is not intended to be limited thereby.

It is to be appreciated that the present invention has applications in any and all methods, procedures and processes involving DNA diagnostics. Examples of such applications include but are not limited to those involving food, water safety, bioterrorism, medical/medicines and/or anything involving pathogen detection. In the food industry, the present invention can be used to monitor the efficacy of preservatives. The method of the invention has the potential to be applied to all cells. Although bacterial cells are exemplified in the example, one of ordinary skill in the art can easily see that the methods of the invention can be applied to many other cell types. The invention can also be used for the identification of substances that can disrupt membranes and/or kill cells, e.g. bacterial cells. The identification of new disinfectants and/or antibiotics are now a priority since multidrug resistance organisms have flourished and spread in health institutions and patients.

It will further be appreciated that the methods of the invention, in combination with quantitative PCR as a tool, can quickly and successfully identify the impact of a disinfectant and/or antibiotic without having to spend time culturing the cells and waiting for growth. In some instances, organisms can take days to weeks to culture, and thus it can take significant time to see if the candidate substance has been able to kill cells, like microorganisms. In other instances, certain organisms will not grow in cell culture, therefore making it difficult to determine if a substance was effective. Thus, applying the novel methods of the invention can save time and resources for identification of novel disinfectants and/or antibiotics.

A further advantage of the novel methods according to the invention is ease of use. For example, using these methods, large amounts of samples can easily be tested for the presence of viable cells, e.g. bacteria. For example, samples may be tested for the presence of potentially live bacteria with intact cell membranes. In another embodiment, environmental samples may be tested for the presence of viable cells, e.g. bacteria. These samples may be, for example, collected from soil or be parts of plants. The methods according to the invention can further be used for testing of treated waste water both before and after release.

The methods according to the invention may further be used for testing medicinal samples, e.g., stool samples, blood cultures, sputum, tissue samples (also cuts), wound material, urine, and samples from the respiratory tract, implants and catheter surfaces.

Another field of application of the methods according to the invention can be the control of foodstuffs. In other embodiments, the food samples are obtained from milk or milk products (yogurt, cheese, sweet cheese, butter, and buttermilk), drinking water, beverages (lemonades, beer, and juices), bakery products or meat products. The method of the invention can determine if preservatives in the food or antimicrobial treatment of food (such as pasteurization) has prevented cell growth. A further field of application of the method according to the invention is the analysis of pharmaceutical and cosmetic products, e.g. ointments, creams, tinctures, juices, solutions, drops, etc.

The methods of the invention solve the problem of long incubation times (in the range of days) making the older methods unsuitable for timely warning and preventive action. In addition, modern PCR based methods can give false positive results (testing positive for an organism although the organism is not viable). Moreover, research has recently discovered that some organisms can, under certain circumstances, lose the ability to replicate although they are still viable. These ‘viable but not culturable’ (VBNC) bacteria cannot be detected using traditional cultivation but might regain their ability to grow if transferred to a more appropriate environment. These drawbacks are solved by applying molecular approaches based on the detection of genetic material/DNA of these organisms in combination with the methods of the invention. Thus, quick and accurate results regarding viable organisms in a sample, e.g. contaminated water, sewage, food, pharmaceuticals and/or cosmetics, can prevent contaminated products from being released to the public. The methods of the invention can save resources, by minimizing false positives (testing positive for a pathogen although the pathogen is not viable) and rapid testing of samples, as compared to the current time consuming methods.

In addition, the methods of the invention can identify potentially viable members of a microbial community for ecological studies, health of specific soils for agricultural and/or ecological systems. Traditionally identifying a bacterial community has been performed using cultivation-based approaches or plate counts. The more colonies that are counted, the more bacteria are estimated to be in the original sample roblems, however, arise from sometimes long incubation times (in the range of days) making this method unsuitable for timely and accurate results. These drawbacks are utilizing the methods of the invention.

Non-limiting examples of bacteria that can be subjected to analysis using the methods of the invention or to detect potential viability in a sample using the method of the invention comprise, in addition to SA as previously described: B. pertussis, Leptospira pomona, S. paratyphi A and B, C. diphtheriae, C. tetani, C. botidinum, C. perfringens, C. feseri and other gas gangrene bacteria, B. anthracis, P. pestis, P. multocida, Neisseria meningitidis, N. gonorrheae, Hemophilus influenzae, Actinomyces {e.g., Norcardia), Acinetobacter, Bacillaceae {e.g., Bacillus anthrasis), Bacteroides {e.g., Bacteroides fragilis), Blastomycosis, Bordetella, Borrelia {e.g., Borrelia burgdorferi), Brucella, Campylobacter, Chlamydia, Coccidioides, Corynebacterium {e.g., Corynebacterium diptheriae), E. coli {e.g., Enterotoxigenic E. coli and Enterohemorrhagic E. coli), Enterobacter (e.g. Enter obacter aerogenes), Enterobacteriaceae (Klebsiella, Salmonella (e.g., Salmonella typhi, Salmonella enteritidis, Serratia, Yersinia, Shigella), Erysipelothrix, Haemophilus (e.g., Haemophilus influenza type B), Helicobacter, Legionella (e.g., Legionella pneumophila), Leptospira, Listeria (e.g., Listeria monocytogenes) Mycoplasma, Mycobacterium (e.g., Mycobacterium leprae and Mycobacterium tuberculosis), Vibrio (e.g., Vibrio cholerae), Pasteurellacea, Proteus, Pseudomonas (e.g., Pseudomonas aeruginosa), Rickettsiaceae, Spirochetes (e.g., Treponema spp., Leptospira spp., Borrelia spp.), Shigella spp., Meningiococcus, Pneumococcus and all Streptococcus (e.g., Streptococcus pneumoniae and Groups A₃ B, and C Streptococci), Ureaplasmas. Treponema pollidum, Staphylococcus aureus, Pasteurella haemolytica, Corynebacterium diptheriae toxoid, Meningococcal polysaccharide, Bordetella pertusis, Streptococcus pneumoniae, Clostridium tetani toxoid, and Mycobacterium bovis. The above list is intended to be merely illustrative and by no means is meant to limit the invention to detection to those particular bacterial organisms.

A particularly preferred embodiment of the present invention utilizes PCR. General procedures for PCR are taught in U.S. Pat. No. 4,683,195 (Mullis, et al.) and U.S. Pat. No. 4,683,202 (Mullis, et al.). However, optimal PCR conditions used for each amplification reaction are generally empirically determined or estimated with computer software commonly employed by artisans in the field. A number of parameters influence the success of a reaction. Among them are annealing temperature and time, extension time, Mg²⁺, pH, and the relative concentration of primers, templates, and deoxyribonucleotides. Generally, the template nucleic acid is denatured by heating to at least about 95° C. for 1 to 10 minutes prior to the polymerase reaction. Approximately 20-99 cycles of amplification are executed using denaturation at a range of 90° C. to 96° C. for 0.05 to 1 minute, annealing at a temperature ranging from 48° C. to 72° C. for 0.05 to 2 minutes, and extension at 68° C. to 75° C. for at least 0.1 minute with an optimal final cycle. In one embodiment, a PCR reaction may contain about 100 ng template nucleic acid, 20 uM of upstream and downstream primers, and 0.05 to 0.5 mm dNTP of each kind, and 0.5 to 5 units of commercially available thermal stable DNA polymerases.

A variation of the conventional PCR is reverse transcription PCR reaction (RT-PCR), in which a reverse transcriptase first coverts RNA molecules to single stranded cDNA molecules, which are then employed as the template for subsequent amplification in the polymerase chain reaction. Isolation of RNA is well known in the art. In carrying out RT-PCR, the reverse transcriptase is generally added to the reaction sample after the target nucleic acid is heat denatured. The reaction is then maintained at a suitable temperature (e.g. 30-45° C.) for a sufficient amount of time (10-60 minutes) to generate the cDNA template before the scheduled cycles of amplification take place. One of ordinary skill in the art will appreciate that if a quantitative result is desired, caution must be taken to use a method that maintains or controls for the relative copies of the amplified nucleic acid. Methods of “quantitative” amplification are well known to those of skill in the art. For example, quantitative PCR can involve simultaneously co-amplifying a known quantity of a control sequence using the same primers. This provides an internal standard that may be used to calibrate the PCR reaction.

Another alternative of PCR is quantitative PCR (qPCR). qPCR can be run by competitive techniques employing an internal homologous control that differs in size from the target by a small insertion or deletion. However, non-competitive and kinetic quantitative PCR may also be used. Combination of real-time, kinetic PCR detection together with an internal homologous control that can be simultaneously detected alongside the target sequences can be advantageous.

Primers for PCR, RT-PCR and/or qPCR are selected within regions or specific bacteria which will only amplify a DNA region which is selected for that specific organism. Alternatively, primers are selected which will hybridize and amplify a section of DNA which is common for all organisms. Primer selection and construction is generally known in the art. In general, one primer is located at each end of the sequence to be amplified. Such primers will normally be between 10 to 35 nucleotides in length and have a preferred length from between 18 to 22 nucleotides. The smallest sequence that can be amplified is approximately 50 nucleotides in length (e.g., a forward and reverse primer, both of 20 nucleotides in length, whose location in the sequences is separated by at least 10 nucleotides). Much longer sequences can be amplified. One primer is called the “forward primer” and is located at the left end of the region to be amplified. The forward primer is identical in sequence to a region in the top strand of the DNA (when a double-stranded DNA is pictured using the convention where the top strand is shown with polarity in the 5′ to 3′ direction). The sequence of the forward primer is such that it hybridizes to the strand of the DNA which is complementary to the top strand of DNA. The other primer is called the “reverse primer” and is located at the right end of the region to be amplified. The sequence of the reverse primer is such that it is complementary in sequence to, i.e., it is the reverse complement of a sequence in, a region in the top strand of the DNA. The reverse primer hybridizes to the top end of the DNA. PCR primers should also be chosen subject to a number of other conditions. PCR primers should be long enough (preferably 10 to 30 nucleotides in length) to minimize hybridization to greater than one region in the template. Primers with long runs of a single base should be avoided, if possible. Primers should preferably have a percent G+C content of between 40 and 60%. If possible, the percent G+C content of the 3′ end of the primer should be higher than the percent G+C content of the 5′ end of the primer. Primers should not contain sequences that can hybridize to another sequence within the primer (i.e., palindromes). Two primers used in the same PCR reaction should not be able to hybridize to one another. Although PCR primers are preferably chosen subject to the recommendations above, it is not necessary that the primers conform to these conditions. Other primers may work, but have a lower chance of yielding good results.

PCR primers that can be used to amplify DNA within a given sequence can be chosen using one of a number of computer programs that are available. Such programs choose primers that are optimum for amplification of a given sequence (i.e., such programs choose primers subject to the conditions stated above, plus other conditions that may maximize the functionality of PCR primers). One computer program is the Genetics Computer Group (GCG recently became Accelrys) analysis package which has a routine for selection of PCR primers.

The oligonucleotide primers and probes disclosed below can be made in a number of ways. One way to make these oligonucleotides is to synthesize them using a commercially-available nucleic acid synthesizer. A variety of such synthesizers exists and is well known to those skilled in the art.

Another alternative of PCR useful in connection with the invention is isothermal nucleic acid amplification assay for the detection of specific DNA or RNA targets. Non-limiting examples for isothermal amplification of nucleic acids are homogeneous real-time strand displacement amplification, Phi29 DNA polymerase based rolling circle amplification of templates for DNA sequencing, rolling-circle amplification of duplex DNA sequences assisted by PNA openers or loop-mediated isothermal amplification of DNA analytes.

Nucleic acid may also be detected by hybridization methods. In these methods, labeled nucleic acid may be added to a substrate containing labeled or unlabeled nucleic acid probes. Alternatively, unlabeled or unlabeled nucleic acid may be added to a substrate containing labeled nucleic acid probes. Hybridization methods are disclosed in, for example, Micro Array Analysis, Marc Schena, John Wiley and Sons, Hoboken N.J. 2003.

Methods of detecting nucleic acids can include the use of a label. For example, radiolabels may be detected using photographic film or a phosphoimager (for detecting and quantifying radioactive phosphate incorporation). Fluorescent markers may be detected and quantified using a photodetector to detect emitted light (see U.S. Pat. No. 5,143,854, for an exemplary apparatus). Enzymatic labels are typically detected by providing the enzyme with a substrate and measuring the reaction product produced by the action of the enzyme on the substrate. Colorimetric labels are detected by simply visualizing the colored label. In one embodiment, the amplified nucleic acid molecules are visualized by directly staining the amplified products with a nucleic acid-intercalating dye. As is apparent to one skilled in the art, exemplary dyes include but not limited to SYBR green, SYBR blue, DAPI, propidium iodine, Hoeste, SYBR gold and ethidium bromide. The amount of luminescent dyes intercalated into the amplified DNA molecules is directly proportional to the amount of the amplified products, which can be conveniently quantified using a Fluorolmager (Molecular Dynamics) or other equivalent devices according to manufacturers' instructions. A variation of such an approach is gel electrophoresis of amplified products followed by staining and visualization of the selected intercalating dye. Alternatively, labeled oligonucleotide hybridization probes (e.g. fluorescent probes such as fluorescent resonance energy transfer (FRET) probes and colorimetric probes) may be used to detect amplification. Where desired, a specific amplification of the genome sequences representative of the biological entity being tested, may be verified by sequencing or demonstrating that the amplified products have the predicted size, exhibit the predicted restriction digestion pattern, or hybridize to the correct cloned nucleotide sequences.

The present invention also comprises kits. For example, the kit can comprise primers useful for amplifying nucleic acid molecule corresponding to organisms specifically or generally, buffers and reagents for isolating DNA, and reagents for PCR. The kit can also include detectably labeled oligonucleotide, which hybridizes to a nucleic acid sequence encoding a polypeptide corresponding to organisms of interest. The kit can also contain a control sample or a series of control samples which can be assayed and compared to a test sample contained. Each component of the kit can be enclosed within an individual container and all of the various containers can be within a single package, along with instructions for interpreting the results of the assays performed using the kit

The contents of all references, patents and published patent applications cited throughout this application, are incorporated herein by reference to the same extent as if each individual publication, patent or patent application was specifically and individually indicated to be incorporated by reference.

The foregoing detailed description has been given for clearness of understanding only and no unnecessary limitations should be understood therefrom as modifications will be obvious to those skilled in the art. It is not an admission that any of the information provided herein is prior art or relevant to the presently claimed inventions, or that any publication specifically or implicitly referenced is prior art.

Unless defined otherwise, all technical and scientific terms used herein have the same meaning as commonly understood by one of ordinary skill in the art to which this invention belongs.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Also, while certain of the preferred embodiments of the present invention have been described and specifically exemplified above, it is not intended that the invention be limited to such embodiments, and any such limitations are contained only in the following claims.

What is claimed is:

1. A method for selectively excluding, from molecular detection, DNA of dead cells from a mixture containing live and dead cells, which method comprises removing dead microbe cell DNA prior to obtaining a positive non contaminated result from a nucleic acid amplification assay thereby indicating that viable cells are present, measuring two or more time points of microbe-specific signal increases from the amplification assay as an indication of the presence of viable microbes, eliminating amplification assay inhibitors from the mixture by the addition of a chemical denaturant, and determining the ratio of live to dead microbes present in the mixture.

2. The method of claim 1, wherein the determination of the ratio of live to dead microbes present in the mixture can be used as a measure of the effectiveness of a therapy or the efficacy of a treatment.

3. The method of claim 1, wherein the chemical denaturant comprises a mixture of one or more chemical agents.

4. The method of claim 1, wherein the amplification assay is a PCR assay.

5. The method of claim 1, wherein the mixture comprises blood and other body fluids.

6. The method of claim 4, wherein performing the PCR assay provides correlation with viable microbe cells from Bacteremia and Fungemia samples for the diagnosis of septicemia.

7. The method of claim 1, wherein signals from killed cells in the mixture are suppressed and membrane-compromised cells in the mixture are excluded from analysis.
